(19) United States
(12) Patent Application Publication
(10) Pub. No.: US 2026/0007803 A1
(43) Pub. Date: Jan. 8, 2026

(54) Method to Treat Osteoporotic Vertebral Body

(71) Applicant: JOY MEDICAL DEVICES CORPORATION, Kaohsiung (TW)

(72) Inventors: Chien-Ping JU, Kansas City, MO (US); Jiin-Huey Chern LIN, Winnetka, IL (US); Chih-Chien WANG, Tainan (TW)

(21) Appl. No.: 18/761,407

(22) Filed: Jul. 2, 2024

(51) Int. Cl. A61L 27/04; A61L 27/12; A61L 27/56

(52) U.S. Cl. CPC A61L 27/047; A61L 27/12; A61L 27/56; A61L 2430/38

(57) ABSTRACT

A percutaneous minimally-invasive procedure to prevent a potential fracture in an osteoporotic vertebra of an osteoporotic patient is provided by minimally-invasively delivering a biocompatible and resorbable porous calcium-based granular material into the osteoporotic vertebral body.

### FIELD OF THE INVENTION

The present invention is related to a minimally invasive method to prevent a potential fracture in an osteoporotic vertebra of an osteoporotic patient who may not have a history of vertebral fracture or may have a history of vertebral fracture and has recovered from a treatment of vertebral fracture.

### BACKGROUND OF THE INVENTION

U.S. Pat. No. 11,399,960 B2 discloses a method for reducing a risk of a potential fracture in an osteoporotic vertebra of an osteoporotic patient comprising identifying an osteoporotic patient who has no history of vertebral fracture, and identifying a vertebra suffering osteoporosis, wherein said osteoporotic vertebra is diagnosed with a T-score lower than −2.5; injecting a calcium-based cement paste into said osteoporotic vertebra, wherein the injected calcium-based cement paste will harden in said osteoporotic vertebra to form a hardened block, and at least a portion of the hardened block is bioresorbable and will be gradually replaced by a newly-grown bone structure in said osteoporotic vertebra, wherein said injecting said calcium-based cement paste into said osteoporotic vertebra comprises drilling a hole on said osteoporotic vertebra, followed by inserting a distal end of a thin tube into said hole so that an empty space created directly by said drilling in said osteoporotic vertebra is fluidly communicated with a proximal end of said thin tube, and feeding said thin tube with said calcium-based cement paste from said proximal end thereof until a desired amount of said calcium-based cement paste leaves said distal end of the thin tube and enters said empty space in said osteoporotic vertebra.

It is the present Inventors' belief that a truly prophylactic treatment to prevent OVCF must be an effective treatment conducted before a vertebral fracture occurring to an osteoporotic vertebra.

### SUMMARY OF THE INVENTION

A method for treating an osteoporotic vertebra in accordance with a preferred embodiment of the present invention comprises implanting a porous calcium-based granular material inside a vertebra body of an osteoporotic vertebra.

Preferably, said implanting comprises drilling a hole into vertebra centrum of the osteoporotic vertebra, and introducing the porous calcium-based granular material into the vertebra centrum through the hole.

Preferably, the hole is drilled on a pedicle of the osteoporotic vertebra. More preferably, the method further comprises drilling another hole on the other pedicle of the osteoporotic vertebra and into the vertebra centrum of the osteoporotic vertebra, and introducing the porous calcium-based granular material into the vertebra centrum through the another hole. Most preferably, the method further comprises drilling a third hole on the vertebra body of the osteoporotic vertebra and into the vertebra centrum of the osteoporotic vertebra, and introducing the porous calcium-based granular material into the vertebra centrum through the third hole.

Alternatively, the hole is drilled on the vertebra body of the osteoporotic vertebra. Preferably, the method further comprises drilling another hole on a pedicle of the osteoporotic vertebra and into the vertebra centrum of the osteoporotic vertebra, and introducing the porous calcium-based granular material into the vertebra centrum through the another hole.

Preferably, said calcium-based granular material implanted into the vertebra centrum of osteoporotic vertebra is at least partially bioresorbable, resulting in a newly-grown bone structure in the vertebra centrum of osteoporotic vertebra, so that a risk of a potential fracture of the osteoporotic vertebra is reduced.

Preferably, said osteoporotic vertebra is diagnosed with a T-score lower than −2.5.

Preferably, said osteoporotic vertebra is substantially in the absence of an endplate fracture.

Preferably, said calcium-based granular material comprises porous calcium-based granules having a porosity of about 30 vol % to about 90 vol % and a granular size of about 0.1 mm to about 2.5 mm, and the porous calcium-based granules comprise calcium phosphate, calcium sulfate, calcium carbonate, calcium oxide, calcium hydroxide, hydroxyapatite, or a combination thereof. More preferably, the porosity is of about 60 vol % to about 80 vol %. More preferably, the granular size is of about 0.5 mm to about 1.5 mm.

Preferably, pores of the porous calcium-based granules have a pore size in a range of about 30 μm to about 300 μm.

Preferably, pores of the porous calcium-based granules comprise interconnected pores.

Preferably, the porous calcium-based granules comprise calcium phosphate, calcium sulfate, hydroxyapatite or a mixture thereof.

Preferably, the porous calcium-based granules comprise a mixture of hydroxyapatite, calcium phosphate and calcium sulfate. More preferably, the calcium phosphate is tetracalcium phosphate (TTCP), dicalcium phosphate (DCP), tricalcium phosphate, monocalcium phosphate or a mixture thereof; and the calcium sulfate is calcium sulfate hemihydrate (CSH), calcium sulfate dihydrate (CSD), anhydrous calcium sulfate, or a mixture thereof. More preferably, the calcium phosphate is tetracalcium phosphate, dicalcium phosphate, or a mixture thereof; and the calcium sulfate is calcium sulfate hemihydrate, calcium sulfate dihydrate, or a mixture thereof.

The present invention also provides a method for reducing a risk of a potential fracture in an osteoporotic vertebra of an osteoporotic patient, which comprises:
identifying a vertebra suffering osteoporosis in said osteoporotic patient, wherein said osteoporotic vertebra is diagnosed with a T-score lower than −2.5; and
implanting a porous calcium-based granular material inside vertebra body of said osteoporotic vertebra, wherein at least a portion of said calcium-based granular material is bioresorbable and will be gradually replaced by a newly-grown bone structure in said vertebra body of said osteoporotic vertebra.

### DETAILED DESCRIPTION OF THE INVENTION

The present invention is related to a minimally invasive method to prevent a potential fracture in an osteoporotic vertebra of an osteoporotic patient, which comprises:
identifying an osteoporotic vertebra;
introducing a porous calcium-based granular material into said osteoporotic vertebra, wherein the introduced porous calcium-based granular material is at least partially bioresorbable and will be gradually replaced by a newly-grown bone structure in said osteoporotic vertebra.

Preferably, said introducing the porous calcium-based granular material into said osteoporotic vertebra comprises drilling a hole on said osteoporotic vertebra, creating a cavity in said osteoporotic through said hole, inserting a distal end of a thin tube into said hole so that the cavity is fluidly communicated with a proximal end of said thin tube, and feeding said thin tube with said porous calcium-based granular material from said proximal end thereof until a desired amount of said porous calcium-based granular material leaves said distal end of the thin tube and enters said cavity.

Preferably, said introducing the porous calcium-based granular material into said osteoporotic vertebra comprises drilling a hole on said osteoporotic vertebra, inserting a distal end of a thin tube into said hole so that an empty space in said osteoporotic vertebra is fluidly communicated with a proximal end of said thin tube, and feeding said thin tube with said porous calcium-based granular material from said proximal end thereof until a desired amount of said porous calcium-based granular material leaves said distal end of the thin tube and enters said empty space in said osteoporotic vertebra.

A suitable tool for delivering the porous calcium-based granular material includes (but not limited to) the tools disclosed in U.S. Pat. Nos. 10,500,383 B2 and 11,166,756 B2.

A porous calcium-based granular material suitable for use in the method of the present invention includes (but not limited to) the porous calcium-based granules disclosed in U.S. Pat. No. 8,784,551 B2. EZECHBONE® Granule is available from JOY MEDICAL DEVICES CORPORATION, 1F., No. 63, Luke 2nd Road, Luzhu District, Kaohsiung City, Taiwan. EZECHBONE® Granule has a particle size of about 0.4-1.2 mm, and a porosity of about 70-80 vol %, and is TTCP/DCPA/CSH-derived porous granules prepared based on the method disclosed in U.S. Pat. No. 8,784,551. The porous granules of calcium compound can be prepared from a mixed powers of TTCP/DCP/calcium sulfate (preferably TTCP/DCPA/CSH; more preferably TTCP/DCPA/CSH/CSD) and a pore forming agent, which is then mixed with a NH⁴⁺ solution to form a paste. The paste is hardened into a block in a mold, followed by washing the pore forming agent from the hardened block and breaking the block into porous granules; or breaking the block prior to the washing. The porous granules are composed of hydroxyapatite, calcium sulfate (primarily CSD), and less significantly unreacted TTCP, DCP, and CSH. (DCPA: dicalcium phosphate anhydrous)

Practically in many cases the osteoporotic vertebra body is so porous that the porous calcium-based granular material can be directly delivered into the osteoporotic vertebral body without need to surgically create a cavity larger than a drilled hole diameter by a cutting tool such as the one disclosed in WO/2019/083784. For an osteoporotic vertebra that is not so porous, using the cutting tool to create a cavity before the delivery can be helpful.

1. A method for treating an osteoporotic vertebra comprising implanting a porous calcium-based granular material inside a vertebra body of an osteoporotic vertebra.

2. The method of claim 1, wherein said implanting comprises drilling a hole into vertebra centrum of the osteoporotic vertebra, and introducing the porous calcium-based granular material into the vertebra centrum through the hole.

3. The method of claim 2, wherein the hole is drilled on a pedicle of the osteoporotic vertebra.

4. The method of claim 3 further comprising drilling another hole on the other pedicle of the osteoporotic vertebra and into the vertebra centrum of the osteoporotic vertebra, and introducing the porous calcium-based granular material into the vertebra centrum through the another hole.

5. The method of claim 4 further comprising drilling a third hole on the vertebra body of the osteoporotic vertebra and into the vertebra centrum of the osteoporotic vertebra, and introducing the porous calcium-based granular material into the vertebra centrum through the third hole.

6. The method of claim 2, wherein the hole is drilled on the vertebra body of the osteoporotic vertebra.

7. The method of claim 6 further comprising drilling another hole on a pedicle of the osteoporotic vertebra and into the vertebra centrum of the osteoporotic vertebra, and introducing the porous calcium-based granular material into the vertebra centrum through the another hole.

8. The method of claim 1, wherein said calcium-based granular material implanted into the vertebra centrum of osteoporotic vertebra is at least partially bioresorbable, resulting in a newly-grown bone structure in the vertebra centrum of osteoporotic vertebra, so that a risk of a potential fracture of the osteoporotic vertebra is reduced.

9. The method of claim 1, wherein said osteoporotic vertebra is diagnosed with a T-score lower than −2.5.

10. The method of claim 1, wherein said osteoporotic vertebra is substantially in the absence of an endplate fracture.

11. The method of claim 1, wherein said calcium-based granular material comprises porous calcium-based granules having a porosity of about 30 vol % to about 90 vol % and a granular size of about 0.1 mm to about 2.5 mm, and the porous calcium-based granules comprise calcium phosphate, calcium sulfate, calcium carbonate, calcium oxide, calcium hydroxide, hydroxyapatite, or a combination thereof.

12. The method of claim 11, wherein the porosity is of about 60 vol % to about 80 vol %.

13. The method of claim 11, wherein the granular size is of about 0.5 mm to about 1.5 mm.

14. The method of claim 11, wherein pores of the porous calcium-based granules have a pore size in a range of about 30 μm to about 300 μm.

15. The method of claim 11, wherein pores of the porous calcium-based granules comprise interconnected pores.

16. The method of claim 11, wherein the porous calcium-based granules comprise calcium phosphate, calcium sulfate, hydroxyapatite or a mixture thereof.

17. The method of claim 11, wherein the porous calcium-based granules comprise a mixture of hydroxyapatite, calcium phosphate and calcium sulfate.

18. The method of claim 17, wherein the calcium phosphate is tetracalcium phosphate, dicalcium phosphate, tricalcium phosphate, monocalcium phosphate or a mixture thereof; and the calcium sulfate is calcium sulfate hemihydrate, calcium sulfate dihydrate, anhydrous calcium sulfate, or a mixture thereof.

19. The method of claim 18, wherein the calcium phosphate is tetracalcium phosphate, dicalcium phosphate, or a mixture thereof; and the calcium sulfate is calcium sulfate hemihydrate, calcium sulfate dihydrate, or a mixture thereof.

20. A method for reducing a risk of a potential fracture in an osteoporotic vertebra of an osteoporotic patient comprising:
identifying a vertebra suffering osteoporosis in said osteoporotic patient, wherein said osteoporotic vertebra is diagnosed with a T-score lower than −2.5; and
implanting a porous calcium-based granular material inside vertebra body of said osteoporotic vertebra, wherein at least a portion of said calcium-based granular material is bioresorbable and will be gradually replaced by a newly-grown bone structure in said vertebra body of said osteoporotic vertebra.
